(19) United States
(12) Patent Application Publication
(10) Pub. No.: US 2022/0409219 A1
(43) Pub. Date: Dec. 29, 2022

(54) ASSEMBLIES AND KITS FOR REAMING, METHODS OF ASSEMBLING REAMERS AND USE THEREOF IN REAMING AN ORTHOPEDIC JOINT

(71) Applicant: DEPUY IRELAND UNLIMITED COMPANY, RINGASKIDDY (IE)

(72) Inventors: Arjun LAMBA, Ringaskiddy - Cork County (GB); Kathiresan RAMU, Zuchwil (CH); Sandesh Hemmuru DEVARAJA, Karnataka (IN); Duncan BEEDALL, LEEDS - YORKSHIRE (GB); James NAYLOR, LEEDS - YORKSHIRE (GB)

(21) Appl. No.: 17/781,145

(22) PCT Filed: Dec. 1, 2020
(86) PCT No.: PCT/EP2020/084167

§ 371 Date: May 31, 2022

(30) Foreign Application Priority Data
Dec. 2, 2019 (IN) 201911049508

(51) Int. Cl. A61B 17/16

(52) U.S. Cl. CPC A61B 17/1659; A61B 17/1624; A61B 2017/00473

(57) ABSTRACT

A reamer head assembly comprising: a base member supporting a reaming member, the reaming member comprising a reaming member configured to ream bone; wherein the reaming head assembly comprises a plurality of recesses arranged about the reaming surface, the plurality of recesses configured to allow manual gripping of the reaming head assembly.

### FIELD OF THE INVENTION

The present invention relates to assemblies for reaming orthopedic joints, kits for reaming orthopedic joints, methods of assembling reamers for reaming orthopedic joints, and methods of preparing reamers for use in surgery of orthopedic joints.

### BACKGROUND TO THE INVENTION

Human and animal bodies have various joints, such as ankles, knees, hips, shoulders and elbows. The joints are formed where two or more skeletal bones meet. Many joints permit movement between those two or more bones. Between the joints of a body that permit motion, typically, cartilage is found.

Cartilage provides lubrication for the motion and also absorbs some of the forces to which a joint is subjected. The cartilage may wear down over time. As a consequence, the bones making up a joint may come into contact leading to pain and reduced joint function.

Other causes of joint damage is arthropathy. Arthropathy, such as arthritis, is a disease of the joint that may lead to conditions such as pain, stiffness and swelling.

A hip joint is one of the joints to may experience degradation or disease. The hip joint is a ball and socket arrangement formed where the femoral head of a femur meets the acetabulum of the pelvis. The femoral head, which is the ball part of the joint, and the acetabulum, which is the socket part of the joint, are coated with cartilage for allowing the femur to articulate relative to the pelvis. Other joints commonly afflicted include those of the spine, knee, shoulder, elbow, carpals, metacarpals, and phalanges of the hand.

An option to treat damaged joints is to replace the parts of the joint that are degraded or diseased with a prosthesis. A commonly used prosthesis is a total joint prosthesis. The total joint prosthesis is used to replace native or natural joint parts with an artificial joint. For example, in a total hip replacement procedure, a natural hip joint may be treated with a total hip replacement prosthesis. The total hip replacement prosthesis includes an artificial femoral part and an artificial acetabular part.

During a surgical procedure to replace a joint, the joint is prepared to receive its respective part using specialized instrumentation. One such instrument is a reamer. Reamers are an instrument used to remove parts of a bone to be replaced and may be used to shape the bone to receive the appropriate prosthetic member.

In a total hip replacement, a reamer may be used to prepare the acetabulum of a recipient to receive a replacement cup prosthesis. Acetabular reamers are typically hemispherical and are used prepare a correspondingly shaped cavity in the acetabulum.

To prepare the cavity, the reamer may be connected to a power tool, which is used to rotate the reamer. The rotating reamer is engaged with the acetabulum and a hemispherical cavity is prepared. The diameter of the reamed cavity is determined by the size of the replacement cup prosthesis that is to be implanted.

For a hip replacement procedure, the diameter of the reamed cavity of the acetabulum may be planned. To reach the planned diameter the physician may start preparing the acetabulum using a reamer with a diameter smaller than planned. The physician may build up to the planned diameter incrementally using reamers with increasing diameters.

In a typical procedure, the smaller diameter reamer would be removed and replaced by a larger diameter reamer. The physician then engages the larger diameter reamer with the acetabulum to ream the cavity to the larger diameter.

### SUMMARY OF THE INVENTION

According to a first aspect of the present invention, there is provided a reamer head assembly configured to allow manual gripping. The manual gripping may improve the way a reamer head assembly can be manipulated.

The reamer head assembly includes a base member and a reaming member. The base member supports the reaming member. The reaming member includes a reaming surface configured to ream bone. The reaming head assembly includes a plurality of recesses arranged about the reaming surface. The plurality of recesses are configured to allow manual gripping of the reaming head assembly.

Each recess of the plurality of recesses may form a grip zone. The grip zone may be free from blades or cutter that are located on the reaming surface and used to ream a bone. The grip zone may facilitate manual gripping of the reamer head assembly by an operator.

Preferably, the reaming member includes a plurality of openings through the reaming surface. The base member defines the plurality of recesses. Each recess of the plurality of recesses is arranged to coincide with an opening of the plurality of openings. The arrangement of the recess and opening to coincide with one another may form a grip zone.

Alternatively, the base member may include a plurality of openings. The plurality of recesses may be indentations located in the reaming member. Each one of the plurality of recesses may be arranged to coincide with an opening of the plurality of openings. Each recess of the plurality of recesses may be seated through an opening of the plurality of openings. Seating of the recesses through the openings may provide a feature through which a torque applied to the base member is transmitted to the reaming member.

Due to the grip zone being free from cutters or blades, no or minimal contact of a finger and thumb with blades or cutters located on the reaming surface takes place when the reamer head assembly is gripped.

At least one recess of the plurality of recesses may include an indication of the size of the reaming head assembly.

The base member may include a seating surface. The seating surface may include an aperture. The aperture may be shaped and configured to receive a coupling of a driver. The seating surface may include an indication of the size of the reaming head assembly.

The base may include a seating surface upon which a support structure is arranged. The support structure may be configured to support the reaming member. The support structure may have a plurality of retaining elements. The reaming member may have a plurality of slots. When the reaming member and base member are assembled, each one of the plurality of retaining elements may be engaged with a slot of the plurality of slots to couple together the base member and the reaming member.

The reaming member may have an external surface of the reaming member. The external surface may have a curvature appropriate to ream a socket of skeletal joint. The curvature may be appropriate to ream a hemispherical cavity in a bone.

The reaming member may have an internal surface. A support structure of the base member may abut the internal surface. Abutment of the internal surface by the support structure minimizes deformation of the reaming member during a reaming procedure.

The base member may include a support structure. The support structure may have a plurality of curved supports shaped to abut the internal surface.

The reaming member may have a plurality of openings. The plurality of openings may pass through the reaming surface. The base member may have a plurality of supports. Each support may define a recess of the plurality of recesses. At least one recess of the plurality of recesses may be arranged to coincide with an opening of the plurality of openings.

The arrangement of the recess and opening to coincide with one another may form a grip zone. The grip zone may be free from cutters or blades and facilitate manual gripping of the reamer head assembly with no or minimal contact of an operator with the cutters or blades located on the reaming surface.

At least one recess of the plurality of recesses arranged in a support may have an indication of the size of the reaming head assembly.

The reaming member and the base may define a cavity. The reaming member may have a plurality of cutters. Each cutter may include a blade on the reaming member. The blade may lead into a channel. The channel may lead into the cavity. The blade may be located on the reaming surface of the reaming member. The channel may direct debris from the reaming surface into the cavity. The debris may be bone or other tissue cut by the blade of the reamer head assembly. The cavity may capture the debris.

According to a second aspect of the present invention, there is provided a method of preparing a reamer for use in a surgical process. The method includes the steps of:

providing a reamer head assembly comprising:
a base member supporting a reaming member, the reaming member comprising a reaming member configured to ream bone; and
a plurality of recesses arranged about the reaming surface, the plurality of recesses configured to allow manual gripping of the reaming head assembly;

providing a reamer driver;

gripping the reamer head assembly using the plurality of recesses; and coupling the reamer head assembly to the reamer driver by manipulation of the reamer head assembly using the plurality of recesses.

According to a third aspect of the present invention, there is provided a method of removing a reamer head assembly from a reamer driver. The method includes the steps of:

providing a reamer driver to which a reamer head assembly is coupled, the reamer head assembly comprising:
a base member supporting a reaming member, the reaming member comprising a reaming member configured to ream bone; and
a plurality of recesses arranged about the reaming surface, the plurality of recesses configured to allow manual gripping of the reaming head assembly; gripping the reamer head assembly using the plurality of recesses; and

decoupling the reamer head assembly from the reamer driver by manipulation of the reamer head assembly using the plurality of recesses.

According to a fourth aspect of the present invention, there is provided a method of using a reamer head assembly during a reaming procedure, the method comprising the steps of:

providing a first reamer head assembly comprising:
a base member supporting a reaming member, the reaming member comprising a reaming member configured to ream bone; and
a plurality of recesses arranged about the reaming surface, the plurality of recesses configured to allow manual gripping of the reaming head assembly; providing a reamer driver;

gripping the first reamer head assembly using the plurality of recesses; and

coupling the first reamer head assembly to the reamer driver by manipulation of the first reamer head assembly using the plurality of recesses;

applying the first reamer head assembly to a bone to be reamed;

using the reamer driver to rotate the first reamer head assembly to thereby ream a cavity in a bone;

after the cavity is reamed in the bone, gripping the first reamer head assembly using the plurality of recesses; and

decoupling the first reamer head assembly from the reamer driver by manipulation of the first reamer head assembly using the plurality of recesses.

Preferably, the method further includes the steps of:

after the first reamer head assembly has been decoupled, providing a second reamer head assembly, the second reamer head assembly comprising:
a base member supporting a reaming member, the reaming member comprising a reaming member configured to ream bone; and
a plurality of recesses arranged about the reaming surface, the plurality of recesses configured to allow manual gripping of the reaming head assembly;

gripping the second reamer head assembly using the plurality of recesses; and

coupling the second reamer head assembly to the reamer driver by manipulation of the second reamer head assembly using the plurality of recesses;

applying the second reamer head assembly to the cavity reamed in the bone by the first reamer head assembly; and

using the reamer driver to rotate the second reamer head assembly to further ream the cavity.

Preferably, the second reamer head assembly has a diameter larger that the first reamer head assembly.

Preferably, the method further includes the steps of:

after the cavity is further reamed in the bone, gripping the second reamer head assembly using the plurality of recesses; and

decoupling the second reamer head assembly from the reamer driver by manipulation of the first reamer head assembly using the plurality of recesses.

Preferably, reamer head assemblies of incrementally increasing sizes are coupled to the reamer driver, used to the ream the cavity, and decoupled from the reamer driver until a cavity of a desired size is prepared in the bone.

### A BRIEF DESCRIPTION OF THE DRAWINGS

For a more complete understanding of the present invention and the advantages thereof, reference is now made to the following description taken in connection with the following drawings, in which:

FIG. 1 shows a plan view of a reamer head assembly of a first embodiment of the present invention;

FIG. 2 shows a perspective view of the reamer head assembly of FIG. 1;

FIG. 3 shows a side view of the reamer head assembly of FIG. 1;

FIG. 4 shows a perspective view depicting assembly of a base member and a reamer member of the reamer head assembly of FIG. 1;

FIG. 5 shows perspective view of a reamer head assembly of a second embodiment of the present invention;

FIG. 6 shows a perspective view of a base member of the reamer head assembly shown in FIG. 5;

FIG. 7 shows a perspective view of a reamer member of the reamer head assembly shown in FIG.

5;

FIG. 8 perspective view depicting assembly of a base member and a reamer member of the reamer head assembly of FIG. 5;

FIG. 9 shows a side view of first and second differently sized reamer head assemblies of the second embodiment of the present invention;

FIG. 10 shows a plan view of a blister pack containing a reamer head assembly shown in FIG. 5

FIG. 11 shows a perspective view of a reamer member of a reamer head assembly of a third embodiment of the present invention;

FIG. 12 shows an exploded perspective view depicting assembly of a reamer head assembly of a fourth embodiment of the present invention;

FIG. 13 shows a sectional view of the reamer head assembly of FIG. 12;

FIG. 14 shows an exploded perspective view depicting assembly of a reamer head assembly of a fifth embodiment of the present invention;

FIG. 15 shows a perspective view depicting assembly of an intermediary member and a reamer member of a reamer head assembly of a sixth embodiment of the present invention;

FIG. 16 shows a view depicting coupling of a base member of a reamer head assembly of the sixth embodiment to a reamer driver and assembly of the combination of the intermediary member and the reamer member shown in FIG. 15 onto the base member;

FIG. 17 shows a front view of a seating member of the base member of the reamer head assembly shown in FIG. 16;

FIG. 18 shows an illustrative view of an operator gripping the reamer head assembly shown in FIG. 5;

FIG. 19 shows the reamer head assembly of FIG. 5 arranged to be engaged with a reamer driver;

FIG. 20 shows the reamer head assembly of FIG. 5 coupled to a reamer driver;

FIG. 21 shows the reamer head assembly of FIG. 5 coupled to a reamer driver and a power tool coupled to the reamer driver, the reamer driver and power tool being gripped by an operator for use.

### DETAILED DESCRIPTION OF THE PREFERRED EMBODIMENTS

The present invention relates to reamers for reaming cavities in a bone of a human or animal subject. The reamer is shaped and dimensioned to prepare a cavity in a bone to receive a prosthesis. For example, the reamer may have a hemispherical shape to form a cavity in a pelvis shaped to receive an acetabular component of a hip prosthesis.

During joint replacement surgery of, a reamer can be used in bone preparation. The reamer is coupled to a reamer driver. During bone preparation, the physician will typically replace the reamer with another one, usually a larger one. This procedure is undertaken so that a physician can start by reaming a small cavity and go up in size until a cavity of a desired size has been formed.

To replace one reamer with another, the reamer is removed from the driver. Exemplary embodiments of the present invention improve how the reamer can be removed from the reamer driver by providing a grip arrangement. The grip arrangement can be easily grasped to facilitate removal of the reamer from the reamer driver.

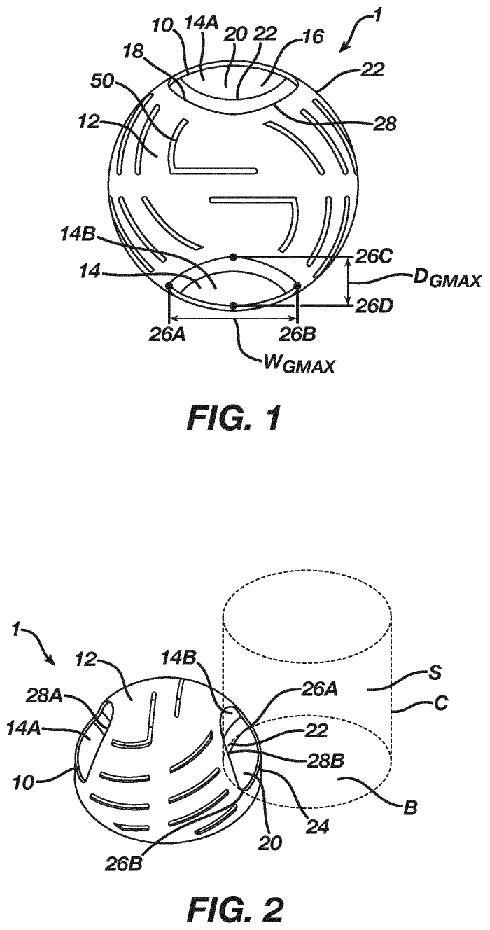

Referring to FIG. 1, a reamer 1 or reamer head assembly according to a first embodiment of the present invention is shown. The reamer 1 has a base member 10, a reaming member 12 and a grip arrangement 14.

The grip arrangement 14 is a defined by a portion of the reamer 1 that can be gripped by a user.

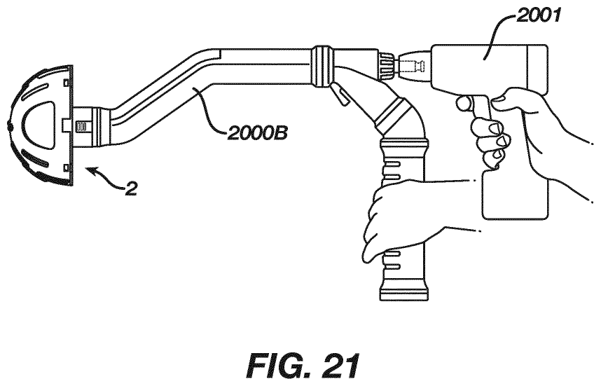

The grip arrangement 14 is free from cutter 50 to thereby facilitate manual gripping and removal of the reamer 1 from the reamer driver 1000 (an exemplary reamer driver is shown in FIG. 21).

The grip arrangement 14 has a first and a second grip zone 14A, 14B. For the reamer 1, the first and second grip zones 14A, 14B are located on either side of the reamer 1.

The grip zones 14A, 14B are Each grip zone 14A, 14B is formed by a combination of a recess 16 in the base member 10 and an aperture 28 in the reaming member 12.

The recess 16 has a floor 20 and a sidewall 22. The floor 20 and the sidewall 22 define the shape and dimensions of a respective grip zone.

Referring to FIG. 2, the overall shape of the grip zone 14A, 14B can be thought of as being defined by removal of a portion of the base 10 and reaming member 12 that intersects with an imaginary cylinder C. In this way, the floor 20 is a portion defined by the base B of the imaginary cylinder C and the sidewall 22 is defined as a portion removed by the curved surface S of the imaginary cylinder C.

The physical shape of a recess 16 of a grip zone 14A, 14B is defined by the intersection of the floor 20 and the sidewall 22 with a perimeter 24 of the base 10 and the reaming member 12. The floor 20 is a flat region or planar surface formed in between the sidewall 22 and the perimeter 24.

The shape of the floor 20 is defined by the sidewall 22 and the perimeter 24. The sidewall 22 is curved. The curvature of the sidewall 22 can be thought of as being defined by the external curved surface S of the imaginary cylinder C. The portion of the sidewall 22 forming the floor 20 arcs between intersection points 26A and 26B at which the sidewall 22 intersects the perimeter 24.

For ease of description the shape of a grip zone 14A, 14B has been defined in connection with an imaginary cylinder. However, as a person of ordinary skill would understand, each grip zone 14A, 14B may have any shape suitable for gripping by a user of the reamer 1.

Referring again to FIG. 1, the floor 20 can be described as being lens shaped. In the embodiment shown, the lens shape is an asymmetrical lens shape. However, as a person of ordinary skill would understand other floor shapes are of course possible.

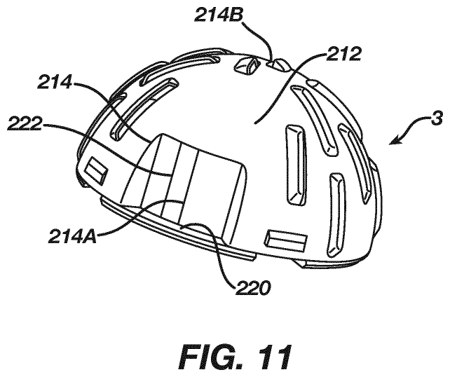

For example, with reference to FIG. 11, a third embodiment of the reamer 3 has a quadrilateral, for example, rectangular, shaped floor 220.

Referring again to FIG. 2, the reaming member 12 has a first aperture 28A through which the grip zone 14A is arranged. The reaming member 12 has a second aperture 28A through which the grip zone 14B is arranged.

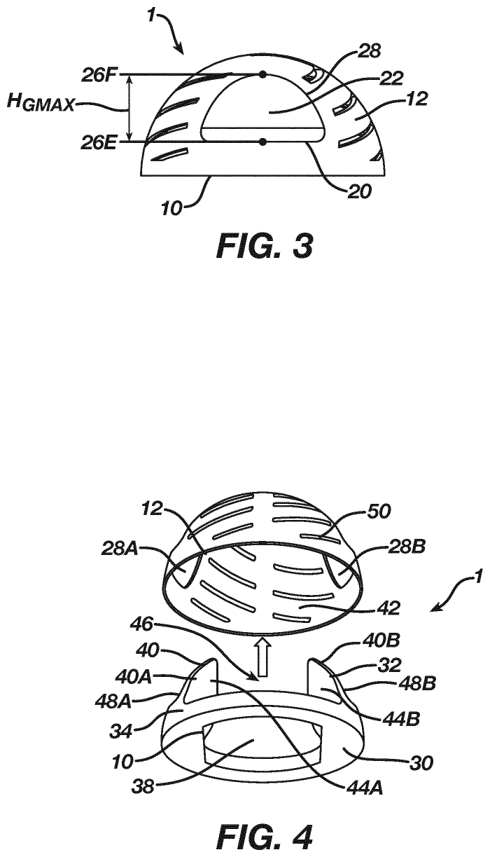

Referring to FIG. 3, a side of the reamer head assembly 1 is shown. The sidewall 22 shown can be thought of as being the sidewall of the grip zone 14A. As can be seen, the sidewall 22 can be described as a wall that extends perpendicular from the floor 20. The grip zone 14B (not shown on FIG. 3) has a shape similar to that of the shape of the grip zone 14A.

The grip zones 14A, 14B are defined by the shape of the floor 20 and the sidewall 22.

The grip zones 14A, 14B are dimensioned to receive a finger or thumb. The dimensions are chosen so that a part of a person used to grip the reamer has no to minimal contact with the reaming member 12.

Each grip zones 14A, 14B has three dimensions: a width, a depth and a height.

As shown by FIG. 1, the width of the grip zones 14A, 14B can thought of in relation to a max width W_{GMAX} of the floor 20. The max width W_{GMAX} can be measured between points 26A, 26B at which the arc of the sidewall 22 intersects with the perimeter 20 of the base 10.

A depth of the floor 20 can be thought of as the distance between the sidewall 22 and the part of the perimeter 20 that form the floor 20 measured perpendicular to an imaginary line (not shown) drawn between intersection points 22, 24. The depth has a maximum depth D_{GMAX} at apexes 26C, 26D. The apexes 26C, 26D are the points on the sidewall 22 and the perimeter 20 that are the furthest apart from each other.

As shown by FIG. 3, the height can be thought of as the height of the sidewall 22. The height of each grip zone 14A, 14B may be measured as the distance between the floor 20 of a grip zone 14A, 14B and a portion of the boundary defining the aperture 28A, 28B that surrounds the sidewall 22. A max height H_{GMAX} is measured between a point 26E, 26F that represents the maximum distance between the floor 20 of the portion of the boundary defining the aperture 28A, 28B that surrounds the sidewall 22.

The dimensions are chosen so that a person's finger or thumb can be positioned in the grip zone 14A, 14B with no to minimal contact with the reaming member 12, particularly the blades or cutters located on the reaming member 12.

The size of grip zone 14A, 14B may differ between different sizes reamer head assemblies 1. For example, in a reaming procedure, a starter reamer, intermediate reamer and final reamer may be used to prepare a bone cavity. These reamers may increase in diameter in 2mm increments. The grip zone 14A, 14B in the smallest reamer head assembly 1, the starter reamer, will have the smallest diameter. The grip zone 14A, 14B of the largest reamer head assembly 1, the final reamer, will have the largest diameter. The approximate width of a grip zone is in the range of 10-30 mms.

As shown by FIG. 4, the reamer 1 is an assembly of a base member 10 and a reaming member 12.

The reaming member 12 has a plurality of cutters 50 spaced apart on the reaming member 12. The plurality of cutters 50 are used to grate or ream bone.

The base member 10 supports the reaming member 12.

The base member 10 has a seating member 30 and a support structure 32.

The seating member 30 has an annular structure. The annular structure is defined by an outer perimeter 34 and an internal wall defining an inner aperture 36. The inner aperture 36 is configured to receive and retain a coupling of a driver, such as a driver 1000 as shown in FIG. 21.

The support structure 32 has a plurality of supports 40. The plurality of supports 40 are shaped to abut the reaming member 12. The abutment of the plurality of supports 40 with the reaming member 12 minimizes deformation of the reaming member 12, in use.

The plurality of supports 40 extend from the seating member 30. Each support 40A, 40B has a truncated dome shape. The dome shape of each support 40A, 40B matches an internal shape of the reaming member 12. The matching of the shape of each support 40A, 40B to the internal shape of the reaming member 12, and resulting abutment of each support 40A, 40B with an internal surface 42 of the reaming member 12, provides a rigidity to the reaming member 12.

In the embodiment shown, the reamer 1 has a first support 40A and a second support 40B. The first and second supports 40A, 40B are arranged on opposite sides of the support structure 32.

Inner sides 44A, 44B of the supports 40A, 40B face each other. The inner sides 44A, 44B are substantially flat. A gap 46 between the inner sides 44A, 44B define a cavity 46 used to capture debris, e.g. bone, removed during the reaming process.

Each support 40A, 40B defines the floor 20 and sidewall 22 of the grip zones 14A, 14B in their outer sides 48A, 48B. The floor 20 and sidewall 22 can be thought of as an indentation in a support 40. The apertures 28A, 28B formed through the reamer member 12 on opposed sides define the outer perimeter of each grip zone 14A, 14B such that when assembled, the floor 20 and sidewall 22 on each support 40A, 40B align with a respective apertures 28A, 28B to form a grip zone 14A, 14B of the grip arrangement 14.

The base member 10 is fabricated from a plastics material. Suitable materials may be a polymer, such as polyacrylamide, polyaryletherketone, polyetheretherkeytone. IXEF and Avaspire are two trade names of forms of polyacrylamide that could be used to fabricate the base. Of course, other materials may be used to fabricate the base member 10.

The reaming member 12 is fabricated from a metal. A suitable metal may be stainless steel. For example, the stainless steel may be type 17-4 PH stainless steel. Of course, other materials may be used to fabricate the reaming member 12.

To assemble the reamer 1, the base member 10 and reaming member 12 are fabricated separately.

The base member 10 is fabricated using injection molding. Of course, any other suitable manufacturing technique may be used.

The reaming member may be fabricated using hydroforming. Of course, any other suitable manufacturing technique may be used.

The outer perimeter 34 and outer sides 48A, 48B of the base member 10 are dimensioned to form an interference fit with the inner surface 42 of the reaming member 12

To assemble the reamer 1, the base member 10 is aligned with the reaming member 12 to align each aperture 28A, 28B is aligned with a respective floor 20 and sidewall 22 of a support 40A, 40B.

As shown by the arrow, the aligned base member 10 is pressed into the aligned reaming member 12 to form the reamer 1. Of course, as a person of skill would understand, the reaming member 12 may be pressed over the base member 10.

The support structure 46 and reaming member 12 are coupled together using an interference fit. The reamer 1 so formed has a generally hemispherical shape featuring indentations formed by the apertures 28A, 28B and the floor 20 and sidewall 22 of each support. The indentations providing grip zones 14A, 14B of the grip arrangement 14. The grip arrangement 14 facilitates manual gripping of the reamer 1 to facilitate its easy removal from a reamer driver 1000.

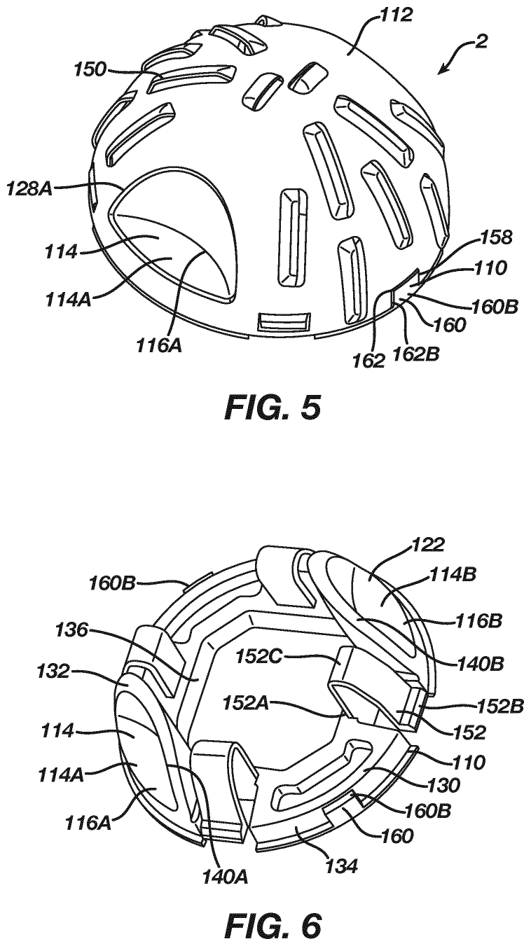

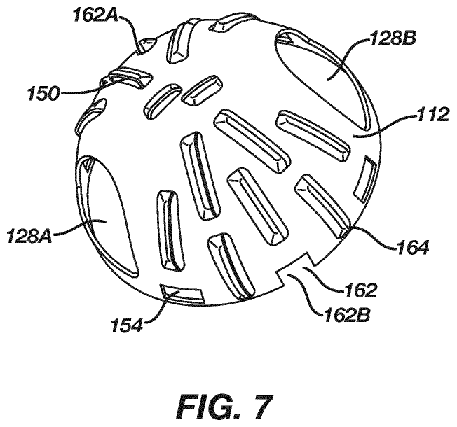

Referring to FIGS. 5-9 a second embodiment of a reamer 2 is shown. The reamer 2 has many features in common with the reamer 1 of the first embodiment as represented by FIGS. 1-4. For ease of reference and simplicity, the description of the reamer 2 describes additional features and alternatives to or variations of the features of reamer 1. For those features that are not described as additional, alternative or variations, a person of skill would understand that those features in reamer 2 are the same or similar to the corresponding features of reamer 1.

Referring to FIG. 5, the reamer 2 has a body member 110, a reamer member 112 and a grip arrangement 114. The grip arrangement 114 facilitates manual gripping and manipulation of the reamer 2.

The reaming member 112 features a plurality of cutters 150. The grip arrangement 114 is a region of the reaming member 112 that is free from cutters. The cutter-free region is dimensioned to enable the reamer 2 to be gripped by, for example, an operator's finger and thumb without said finger or thumb contacting a cutter 150.

Referring to FIGS. 5-7, similarly to the reamer 1, the grip arrangement 114 of the reamer 2 has a first and a second grip zone 114A, 114B.

First and second apertures 128A, 128B are formed through the reamer member 112.

A first and a second recess 116A, 116B is located in the base member 110.

The first and second grip zones are defined by a respective recess 116A, 116B that is aligned with a respective aperture 128A, 128B.

The first and second grip zones 114A, 114B are a cutter-free region and are arranged to enable gripping of the reamer 2 by, for example, an operator's finger and thumb without said finger or thumb contacting a cutter 150.

The first and second grip zones 114A, 114B are located on opposite sides of the base member 10. The grip zones 114A, 114B can be manually grasped to facilitate removal of the reamer 2 from the reamer driver 1000 (an exemplary reamer driver is shown in FIG. 21). Of course, other arrangements of grip zones are possible, as a person of skill in the art would understand. For example, the reamer 2 can have any number of grip zones, for example three grip zones or four grip zones. Grip zones may be clustered together on one side of the reamer to receive one or more fingers, with a further grip zone located at a different location to receive a thumb. Each grip zone may include a plurality of clustered recesses. Other variations and alternatives are of course possible as a person of skill would understand.

The base member 110 has a seating member 130 and a support structure 132.

The seating member 130 has an annular structure. The annular structure is defined by an outer perimeter 134 and an internal wall defining an inner aperture 136. The inner aperture 136 is configured to receive and retain a coupling of a driver, such as a driver 1000 as shown in FIG. 21.

The reamer 2 has a coupling arrangement 152, 154 for coupling the reamer member 112 to the base member 110.

The coupling arrangement 152, 154 has a plurality of resilient members 152 provided on the seating member 130 and a plurality of slots 154 located on the reaming member 112. The plurality of slots 154 are located to matingly receive the plurality of resilient members 152. With each one of the plurality of resilient members 152 located in a respective one of the plurality of slots 154, the reaming member 112 is coupled to the base member 110.

The resilient member 152 is a snap feature. The resilient member has a flexural strength sufficient to retain the connection of the reaming member 112 to the base member 110 during a reaming procedure.

The resilient member 152 extends from a proximal end 152A to a distal end 152B. The proximal end 152A is located on the seating member 130. The distal end 152B defines a foot. The foot 152B is shaped to engage with the slot 154.

From the proximal end 152A, the resilient member 152 extends away from the seating member 130 to a resilient joint 152C. The joint 152C defines a transition zone of the resilient member 152. At the joint 152C, the resilient member 152 curves to transition from extending away from to extending towards the seating member 130.

Each foot 152B of the plurality of resilient members 152 is locatable in a respective one of the plurality of slots 154 to couple the reaming member 112 to the base member 110.

The reamer 2 has an alignment mechanism 158. The alignment mechanism 158 has a first member 160 located on the seating member 130 and a second member 162 located on the reaming member 112.

The first member 160 has a first and a second protrusion 160A, 160B. Each protrusion 160A, 160B is located on the outer perimeter 134. The first and second protrusion 160A, 160B are located on opposed sides of the seating member 130. Each one of the first and second protrusions 160A, 160B are provided in a region of the seating member located in between the first and second supports 140A, 140B.

The second member 162 is a first and a second alignment recess 162A, 1628 located in an edge 164 of the reaming member 112.

The first and second alignment recesses 162A, 162B are located in the edge 164 to matingly receive the first and a second protrusions 160A, 160B. When assembled, with the first and a second protrusions 160A, 160B located in the first and a second alignment recesses 162A, 162B, the apertures 128A, 1288 and cavities 116A, 116B are aligned with each other and defines the grip zones 114A, 114B.

The combination of the coupling arrangement 152, 154 and the alignment mechanism 158 provides a mechanism through which torque is transmitted by the base member 110 from the drive 1000 to the reaming member 112.

The base member 110 is fabricated from a plastics material. Suitable materials may be a polymer, such as polyacrylamide, polyaryletherketone, polyetheretherkeytone. IXEF and Avaspire are two trade names of forms of polyacrylamide that could be used to fabricate the base member 110. Of course, other materials may be used to fabricate the base member 110.

The reaming member 12 is fabricated from a metal. A suitable metal may be stainless steel. For example, the stainless steel may be type 17-4 PH stainless steel. Of course, other materials may be used to fabricate the reaming member 112. The base member 110 and reaming member 112 are fabricated separately.

The base member 110 is fabricated using injection molding. Of course, any other suitable manufacturing technique may be used.

The reaming member 112 may be fabricated using hydroforming. Of course, any other suitable manufacturing technique may be used.

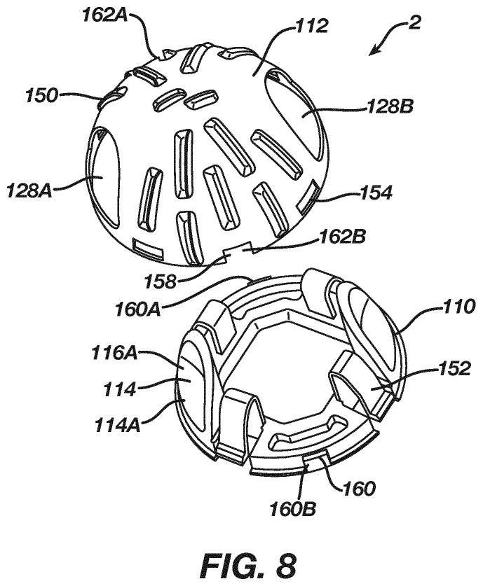

Referring to FIG. 8, to assemble the reamer 2 the base member 110 is aligned with the reaming member 112. In the aligned configuration, the first member 160 is aligned with the second member 162 of the alignment mechanism 158.

With the first and second protrusions 160A, 160B located to be received by the first and second alignment recesses 162A, 162B, the base member 110 and the reaming member 112 are engaged with each by pressing them together.

The base member 110 and the reaming member 112 are pressed together until the each one of the plurality of resilient members 152 is engaged with a respective one of the plurality of slots 154.

With the plurality of resilient members 152 engaged with a respective one of the plurality of slots 154 and the first and second protrusions 160A, 160B located in the first and second alignment recesses 162A, 162B, a reamer head assembly 2 is formed in which a first and a second grip zones 114A, 114B free from cutters 150. The grip zones 114A, 114B facilitate manual gripping of the reamer head assembly 2 to facilitate easy removal from a reamer driver 1000.

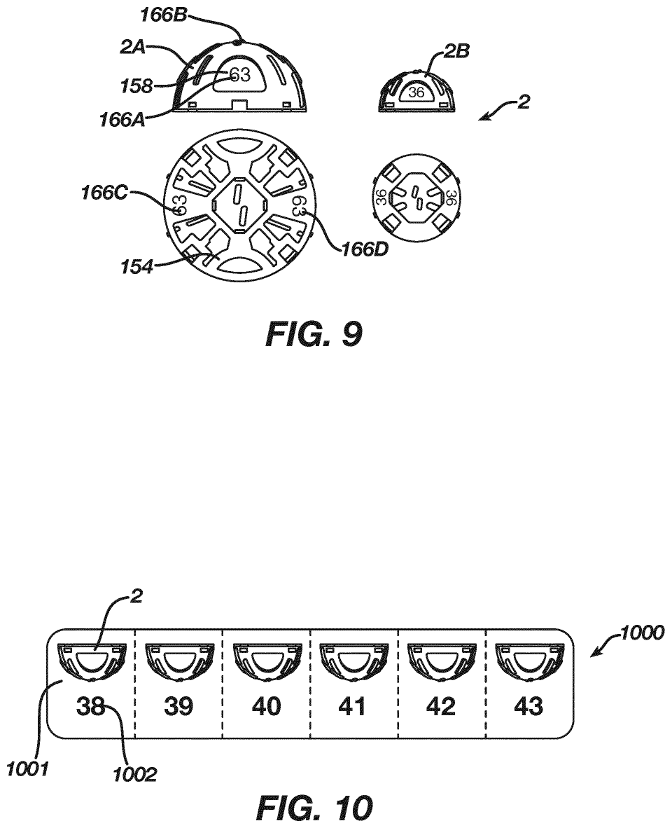

FIG. 9 shows exemplary version of the reamer 2 of different sizes.

The reamer 2 has an indication 166. The indication 166 informs a user of the size of the cavity that is expected to be formed in a pelvis through reaming with the reamer 2.

In the image on left in FIG. 9, a reamer 2A features a marking of the number “63”. In the image on the right in FIG. 9, a reamer 2B features a marking of the number “36”. These numbers inform a user of a size of the reamer 2. The size is the diameter in millimeters of the cavity that is expected to be formed in a pelvis through reaming with the reamer 2.

In the embodiment shown, the indication 166 has a plurality of indicia 166A, 166B, 166C, 166D. Each indicium 166A, 166B, 166C, 166D is visible to a user. The indicia 166A, 166B are located in grip zones 114A, 114B of a grip arrangement 114. The indicia 166C, 166D are located on an external surface 154 of the base member 110. The external surface 166 is visible post-assembly of the reamer 2.

As person of skill would understand, the indication 166 may be added to the base member 110 before or after the base member is used to form a reamer head assembly 2 as described with reference to FIG. 8. The indication 166 may be marked using any suitable technique. For example, the indication 166 may be laser etched onto the or each grip zone 114A, 114B. An exemplary laser printing or etching technique is CO2 laser printing.

Referring to FIG. 10, in a reaming procedure, several different sizes of reamer 2 may be used. Post assembly, each reamer 2 may be individually packed in a different portion of the blister pack 1000.

The blister pack 1000 has six separate bays 1001 containing different sizes of reamer head assemblies 2. As shown by indications 1002, the blister pack contains size 38, 39, 40, 41, 42 and 43 reamer head assemblies. During a reaming procedure, a reaming head assembly may be individually removed from its respective bay 1002 as required and used for reaming a bone. To minimize contact between the person removing the reamer head assembly 2 from its bay 1002, the reamer head assembly 2 is held in the bay in an orientation in which the person can directly grip the grip zones 114A, 114B.

With reference to FIG. 11, a reamer according to a third embodiment of the present invention is shown. A reamer 3 has a reaming member 212 featuring differently shaped grip zones 214A, 214B when compared to the grip zones 114A, 114B of the reamer 2. Referring back to FIG. 5, a grip zone 114A of the grip arrangement 114 is viewed from the side, it may be described as having a semi-circular or semi-ellipsoidal shape. With reference to FIG. 11., the reamer 3 has a quadrilateral, for example square, shaped grip zone 214A, 214B. A person of skill would of course understand other variations of the shape of a grip zone are of course possible.

The reamer 3 has a quadrilateral shaped sidewall 222 and a quadrilateral shaped floor 220 that defines the shape of the grip zones 214A, 214B.

In contrast to the reamer 2, the reamer 3 the grip zones 214A, 214B are formed by the reaming member 212. Since the reaming member 212 is fabricated from a metal material, such as stainless steel. The grip zones 214A, 214B of the reamer 3 are also fabricated of metal using a metal injection molding process.

All other features of the reamer 3 are identical the features of the reamer 2. For ease of reference and simplicity, no further description is provided herein a person of skill would understand how the features of the reamer 2 relate to the reamer 3.

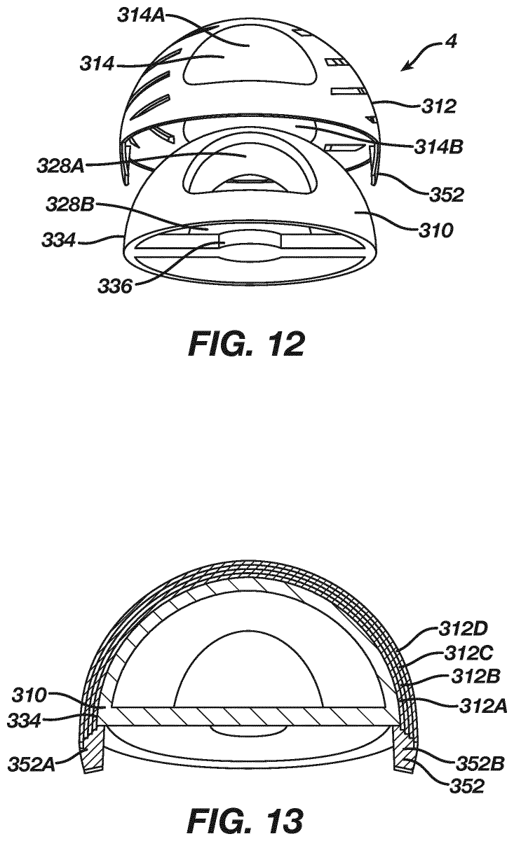

Referring to FIGS. 12 a fourth embodiment of a reamer 4 is shown. In contrast to the first and second embodiments, the reamer 4 has a grip arrangement 314 in which a plurality of apertures 328A, 328B are located in a hemispherical base member 310. Unlike the reamers 1 and 2, where the aperture 28A, 28B, 128A, 128B defines a perimeter of the grip zone 14A, 14B, 114A, 114B, the first and second apertures 328A, 328B define a seating feature through which an internal surface of a grip zone 314A, 314B fits.

Similarly to the reamer 3 of the third embodiment, a reaming member 312 defines the grip zones 314A, 314B.

The reamer 4 features a cross-bar reaming coupling 336.

Referring to FIG. 13, in contrast to the reamers 1 through 3 of the first through third embodiments in which a single reaming member 12, 112, 212 is coupled to a base member 10, 110, 210, the reamer 4 has a plurality of stackable reaming members 312A, 312B, 312C, 312D.

In the exemplary depiction shown by FIG. 13, the reamer 4 has four stackable reaming members 312A, 312B, 312C, 312D.

The first reaming member 312A is positioned over the base member and the grip zones 314A, 314B are located through the apertures 328A, 328B. Each successive reaming member 312B, 312C, 312D, is positioned over the underlying stack of reaming members 312A that is already in situ. The grip zones 314A, 314B of the already in situ reaming members provides the seating feature for the grip zone of the reaming member being positioned on the stack.

In use, the reamer 4 is coupled to a drive, such as a drive 1000 as shown by FIG. 21. Torque is applied to the base member 310 by the drive 1000. Due to the location of the grip zones 314A, 314B through the apertures 328A, 3288, the torque is transmitted to the reaming member 312A or stack of reaming members 312A, 3128, 312C, 312D.

Similarly to the reamers 2 and 3, the base member 310 and reaming members 312A, 3128, 312C, 312D are coupled together with a resilient member 352. The reamers 2 and 3 have resilient members 152 located on the base 110, 210. In contrast, the resilient member 352 is located on the reamer member 312.

Each reaming member 312A, 3128, 312C, 312D has a resilient member 352 that couples each reaming member 312A, 3128, 312C, 312D to the base member 310. The resilient member 352 is a resilient fastener that clips onto an outer perimeter 334 of the base member 310 or, where the reaming member is being positioned over an already in situ reaming member, into a recess 352A located in a resilient member of the underlying stack of reaming members 312A, 3128, 312C, 312D.

Each reaming member 312A, 3128, 312C, 312D has a first and second resilient member 352A, 3528 arranged on opposed sides. Clipping of the first and second resilient member 352A, 3528 to the base member 310 or underlying stack of reaming members 312A, 3128, 312C, 312D fastens that reaming member 312A, 3128, 312C, 312D to the reamer 4.

A first reaming member 312A having a first diameter is positioned on the base member 310 to form a reamer 4 having a first outer diameter of 36 mm. Each successive reaming member 3128, 312C, 312D increases the outer diameter of the reamer 4 by 1 mm. Due to the ability of the reamer 4 to be configured to have successively size reamer diameters, a benefit of an operator being able to ream cavities in a bone of increasing size, where said operator only needs to remove the reamer 4 when a cavity of an appropriate size has been reamed, may be provided.

All other features of the reamer 4 are the same as the reamer 1.

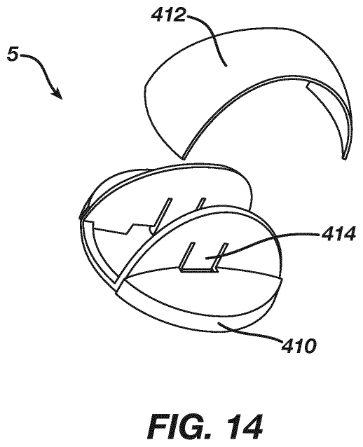

Referring to FIG. 14, a fifth embodiment of the reamer 5 is shown.

In contrast to the reamers 1 through 4 of the first through fourth embodiments of the present invention, the entirety of the grip arrangement 114 is defined by the base member 410.

In contrast to the reamers 1 through 4 of the first through fourth embodiments of the present invention, where the reaming members 12, 112, 212, 312 are substantially hemispherical, the reaming member 412 is a curved band which is form fitted to the base member 410.

All other features of the reamer 5 are substantially the same as those of reamer 1.

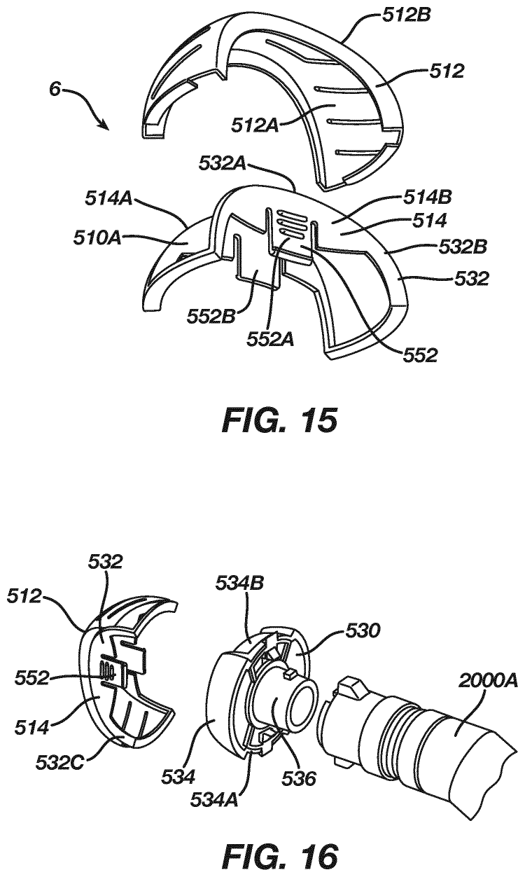

Referring to FIGS. 15 and 16, a sixth embodiment of a reamer 6 is shown. The reamer 6 has a base member 510, a reaming member 512 and a grip arrangement 514.

The base member 510 is a two-part construction.

The base member 510 has a first part and a second part. The first part is a support member 532. The second part is a seating member 530.

A first portion 532A of the support member 532 is shaped and dimensioned to matingly receive the reaming member 512.

The reaming member 512 is a curved band. The reaming member 512 has a plurality of cutters arranged to ream a bone. The reaming member has an internal surface 512A and an external surface 512B. The internal surface 512A sits on the first portion 532A. The external surface SUB is a bone engaging surface.

The first portion 532A is shaped such that it abuts the internal surface 512A. The first portion 532A provides rigidity and support to the reaming member 512.

A second portion 532B of the support member 510B provides rigidity to the first portion 532A and defines an area free from cutters. The area free from cutters defines the grip zones 514A, 514B of the reamer 6.

The support member 532 has a connector 552. The connector 552 is provided to releasably couple the support member 532 to the seating member 530.

The connector 552 has a first and a second connection member 552A. 552B. The connection members 552A. 552B are located in the second portion 532B. The first and second connection members 552A. 552B extend from a midpoint of the reaming member second portion 532B in a direction away from the first portion 532A.

As can be seen by FIG. 16, the support member 532 and the reaming member 512 are assembled together to provide a unitary body. The combination of the support member 532 and reaming member 512 also includes the grip arrangement 514.

If the connection members 552A. 552B are ignored, the combination of the support member 532 and the reaming member 512 define a substantially C-shaped body. Taking the resilient members 513A, 513B into account, and when viewing its elongate side, the overall shape of the combination of the support member 532 and the reaming member 512 has a C-shape with a line extending from the center or, put another way, a curved E-shape.

The combination of the reaming member 512 and support member 532 is couplable to the seating member 530.

The combination of the reaming member 512 and support member 532 is couplable to the seating member 530 using the connector 552.

The first and a second connection member 552A. 552B resiliently couple the combination of the reaming member 512 and support member 532 to the seating member 530.

The seating member 530 has an outer perimeter 534 defining a walled disc-like or annular shaped body. The outer perimeter 534 defines a first and a second seating portion 534A, 534B. The seating portions 534A, 534B receives the support member 532. In use, when the reamer 6 has been assembled and is coupled to be rotated by a drive 2000A, the seating members 534A, 534B in combination with the connector 552 enable a torque applied to the seating member 530 to be transmitted to the support member 532.

The first and second seating portions 534A, 534B are grooves in the outer perimeter 534 for receiving first and second end portions 532C, 532D of the support member 532. The seating portions 534A, 534B are dimensioned to form a press-fit with the first and second end portions 532C, 532D.

The seating member 530 has a coupling 536. The coupling 536 is configured to releasably couple the reamer 6 to the driver 2000A.

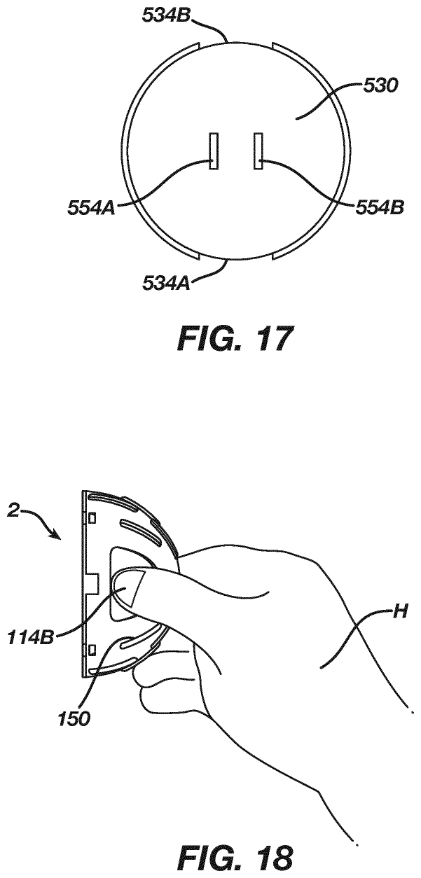

Referring to FIG. 17, the seating member 530 has first and second slots 554A, 554B for receiving the first and second connection members 552A, 552B.

The support member 532 is coupled to the seating member 530 through insertion of the first and second connection members 552A. 552B in respective first and second slots 554A. 554B. The coupling of the first and second connection member 552A. 5528 to the first and second slots 554A. 5548 in combination of the press fit of the first and second seating portions 534A, 5348 and the first and second end portions 532C, 532D enables a torque applied to the seating member 530 to be transmitted to the support member 532.

Referring again to FIG. 16, the reamer 6 may be coupled to a drive 2000A in one of two ways.

In a first approach to coupling the reamer 6 to a drive 2000A, the seating member 530 is first coupled to the drive 2000A. The combination of support member 532 and reaming member 512 are subsequently coupled to the seating member 530 through an operator gripping the grip arrangement 514, positioning the combination and coupling the combination of support member 532 and reaming member 512 to the seating member 530.

In a second approach to coupling the reamer 6 to a drive 2000A, the combination of support member 532 and reaming member 512 are coupled to the seating member 530 through an operator gripping the grip arrangement 514, positioning the combination and coupling the combination of support member 532 and reaming member 512 to the seating member 530 to form an assembled reamer 6. Subsequently, the reamer 6 is coupled to the drive 2000A through an operator gripping the grip arrangement 514 and coupling the reamer 6 to the drive 2000A.

Referring to FIGS. 18 through 21, an exemplary use of a reamer head assembly 2 will now be described.

The reamer head assembly 2 is used in a reaming procedure.

In a first step, a blister pack 1000 containing a plurality of reamer head assemblies 2 of differing sizes is placed in a convenient location in an operating room.

A physician or other suitable person assesses the size of reamer head assembly to be used and requests a certain size.

The person who will couple the reamer head assembly to a driver 2000B looks at the indications 1002 on the blister pack 1000 and opens the appropriate bay 1001.

Due to the orientation of the reamer head assembly 2, the grip zones 114A, 114B are gripped directly.

With reference to FIG. 18, the reamer head assembly 2 with a first diameter is removed from its bay 1001 by manipulation of the reamer head assembly using the grip zones 114A, 114B. With the grip zones 114A, 114B gripped, the reamer head assembly 2 can be moved without fingers or thumbs contacting the cutters 150.

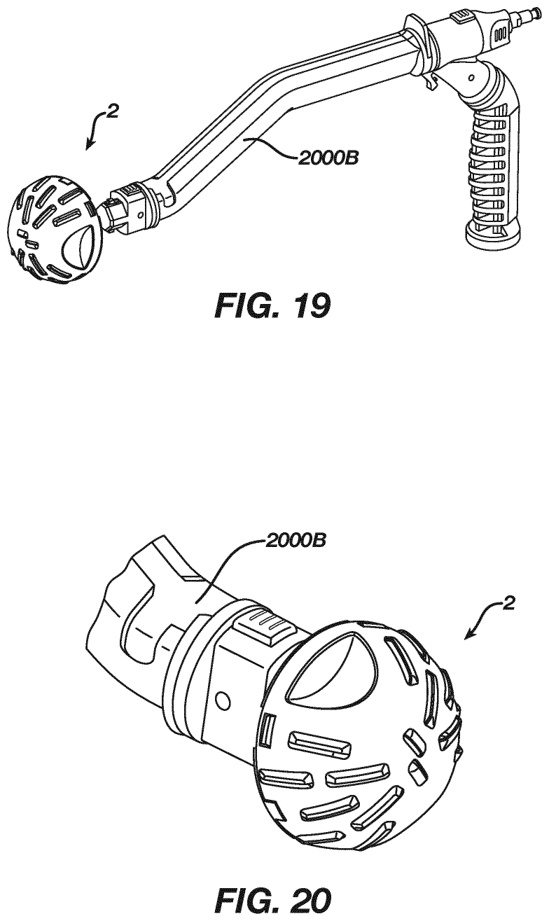

With reference to FIG. 19, which for ease of reference does not show the hand of the person gripping the reamer head assembly 2, the reamer head assembly 2 is oriented to be coupled to the driver 2000B.

With reference to FIG. 20, which for ease of reference does not show the hand H of the person gripping the reamer head assembly 2, the reamer head 2 is coupled to the driver 2000B. The reamer head assembly 2 may now be used to ream a cavity in a bone.

The driver 2000B with the reamer head assembly 2 in situ is passed to the physician. The physician positions the reamer head assembly in a desired location.

With reference to FIG. 21 a power tool 2001 is coupled to the driver 2000B by the physician. The power tool 2001 is used to provide a torque to the driver 2000B to rotate the reamer head assembly 2. For ease of reference, the bone which is to be reamed is not shown in FIG. 21. However, as a person of skill would understand, the reamer head assembly rotatingly driven by the torque applied to the driver 2000B by the power tool 2001 is used to ream a cavity in a bone have a first diameter.

Once the physician has finished with the reaming the cavity to the first diameter, the size of the cavity may be assessed. If it is determined that a larger cavity is required, the reamer head assembly 2 with the first diameter is removed, and a reamer head assembly with a second, larger diameter is coupled to the driver 2000B.

The reamer head assembly 2 with the second diameter is used as described for the reamer head assembly 2 with the first diameter.

The steps described with reference to FIGS. 18 through 21 are repeated with until a cavity of an appropriate size is formed in the bone.

After the reaming procedure is complete, the physician can move on to the next step in the surgical procedure.

As a person of skill would be understand, the exemplary use of the reamer head assembly described by reference to the reamer head assembly 2 and FIGS. 18 through 21 applies equally, with the appropriate variations, to the manner in which the other embodiments of reamer head assemblies 1, 3, 4, 5 and 6 of the present invention may be used.

With reference to FIGS. 12 and 13, a variation may include, for example, coupling a reamer member 312 on top of an in-situ stack of reamer members to increase the size of the reamer head assembly 4, rather than removing a reamer head assembly and coupling a larger reamer head assembly to the reamer driver 2000B.

With reference to FIG. 16, a variation may include, for example, decoupling the support member 532 from the seating member 530 thereby leaving the seating member 530 coupled to the reamer driver 2000A. Following the decoupling, a larger support member 532 with a reaming member 512 having an increased diameter may be coupled to the support member 530 to form a reamer head assembly 6 having a larger diameter, which may be used to ream a larger bone cavity.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

Although preferred embodiments of the present invention have been shown and described, it will be appreciated by those skilled in the art that changes may be made without departing from the scope of the invention as defined in the claims.

In this specification, the terms “comprise”, “comprises”, “comprising” or similar terms are intended to mean a non-exclusive inclusion, such that a system, method or apparatus that comprises a list of elements does not include those elements solely, but may well include other elements not listed.

The reference to any prior art in this specification is not, and should not be taken as, an acknowledgement or any form of suggestion that the prior art forms part of the common general knowledge.

It will of course be understood that this description is by way of example only; alterations and modifications may be made to the described embodiment without departing from the scope of the invention as defined in the claims.

1. A reamer head assembly comprising:
a base member supporting a reaming member, the reaming member comprising a reaming member configured to ream bone;
wherein the reaming had assembly comprises a plurality of recesses arranged about the reaming surface, the plurality of recesses configured to allow manual gripping of the reaming head assembly.

2. The reamer head assembly of claim 1, wherein the base member comprises a plurality of openings, and the plurality of recesses are indentations in the reaming surface;
wherein each one of the plurality of recesses is arranged to coincide with an opening of the plurality of openings.

3. The reamer head assembly of claim 1, wherein the reaming member comprises a plurality of openings passing through the reaming surface; and the base member defines the plurality of recesses; and
wherein each recess of the plurality of recesses is arranged to coincided with an opening of the plurality of openings.

4. The reaming head assembly of claim 1, wherein at least one recess of the plurality of recesses comprises an indication of the size of the reaming head assembly.

5. The reaming head assembly of claim 1, wherein the base member comprises a seating surface defining an aperture through which a drive coupling is arranged, the seating surface comprising an indication of the size of the reaming head assembly.

6. The reaming head assembly of claim 1, wherein the base has a seating surface upon which a support structure is arranged;
wherein the support structure is configured to support the reaming member.

7. The reaming head assembly of claim 6, wherein the support structure comprises a plurality of retaining elements and the reaming member comprises a plurality of slots; and
wherein each one of the plurality of retaining elements is engaged with a slot of the plurality of slots to couple together the base member and the reaming member.

8. The reaming head assembly of claim 6, wherein the reaming member is an external surface of the reaming member and has a curvature to ream a socket of skeletal joint;
wherein an internal surface of the reaming member abuts the supports structure; and
wherein abutment of the internal surface by the support structure minimizes deformation of the reaming member during a reaming procedure.

9. The reaming head assembly of claim 8, wherein the support structure comprises a plurality of curved supports shaped to abut the internal surface.

10. The reaming head assembly of claim 9, wherein the reaming member comprises a plurality of openings passing through the reaming surface;
wherein the supports define the plurality of recesses; and
wherein at least one recess of the plurality of recesses is arranged to coincide with an opening of the plurality of openings.

11. The reaming head assembly of claim 10, wherein at least one recess of the plurality of recesses comprises an indication of the size of the reaming head assembly.

12. The reaming head assembly of claim 1, wherein the reaming member and the base define a cavity, and wherein
the reaming member comprises a plurality of cutters, each cutter comprising a blade on the reaming member that leads into a channel through the reaming member, the channel leading into the cavity.

13. A method of preparing a reamer for use in a surgical process, the method comprising the steps of:
providing a reamer head assembly comprising:
a base member supporting a reaming member, the reaming member comprising a reaming member configured to ream bone; and
a plurality of recesses arranged about the reaming surface the plurality of recesses configured to allow manual gripping of the reaming head assembly;
providing a reamer driver;
gripping the reamer head assembly using the plurality of recesses; and
coupling the reamer hod assembly to the reamer driver manipulation of the reamer head assembly using the plurality of recesses.

14. A method of removing a. reamer head assembly from a reamer driver, the method comprising, the steps of:
providing a reamer driver to which a reamer head assembly is coupled, the reamer head assembly comprising:
a base member supporting a reaming member, the reaming member comprising a reaming member configured to ream bone; and
a plurality of recesses arranged about the reaming sort tee, the plurality of recesses configured to allow manual gripping of the reaming head assembly;
gripping the reamer head assembly using the plurality of recesses; and
decoupling the reamer head assembly from the reamer driver by manipulation of the reamer head assembly using the plurality of recesses.

15. A method of using a reamer head assembly during a reaming procedure, the method comprising the steps of:
providing a first reamer head at comprising;
a base member supporting a reaming member, the reaming member comprising a reaming member configured to ream bone; and
a plurality of recesses arranged about the reaming surface, the plurality of recesses configured to allow manual gripping of the reaming head assembly;
providing a reamer driver;
gripping the first reamer head assembly using the plurality of recesses: and
coupling the first reamer head assembly to the reamer driver by manipulation of the first reamer head assembly using the plurality of recesses;
applying the first reamer head assembly to a bone to be reamed:
using the reamer driver to relate the first reamer head assembly to thereby ream a cavity in a bone;
after the cavity is reamed in the bone, gripping the first reamer head assembly using the plurality of recesses; and
decoupling the first reamer head assembly from the reamer driver by manipulation of the first reamer head assembly using the plurality of recesses.

16. The method of claim 15, further comprising:
after the first reamer head assembly has been decoupled, providing a second reamer head assembly, the second reamer head assembly comprising:
a base member supporting a reaming member, the reaming member comprising a reaming member configured to ream bone; and
a plurality of recesses arranged about the reaming surface, the plurality of recesses configured to allow manual gripping of the reaming head assembly;
gripping the second reamer head assembly using the plurality of recesses: and coupling the second reamer head assembly to the reamer driver by manipulation of the second reamer head assembly using the plurality of recesses;
applying the second reamer head assembly to the cavity reamed in the bone by the first reamer head assembly; and
using the reamer driver to rotate the second reamer head assembly to further ream the cavity.

17. The method of claim 16, wherein the second reamer head assembly has a diameter larger that the first reamer head assembly.

18. The method of claim 16, further comprising:
after the cavity is further reamed the hone, gripping the second reamer head assembly using the plurality of recesses; and
decoupling the second reamer head assembly from the reamer driver by manipulation of the first reamer head assembly using the plurality of recesses.

19. The method of claim 16, wherein reamer head assemblies of incrementally increasing sizes are coupled to the reamer driver, used to the ream the cavity, and decoupled from the reamer driver until a cavity of a desired size is prepared in the bone.
